(19) United States
(12) Patent Application Publication
(10) Pub. No.: US 2026/0002921 A1
(43) Pub. Date: Jan. 1, 2026

(54) METHOD FOR IDENTIFYING PRODUCING AREA OF RIZHAO GREEN TEA

(71) Applicants: Shandong Academy of Agricultural Sciences, Jinan (CN); Rizhao City Lanshan District New Rural Construction and Development Co., Ltd., Rizhao (CN)

(72) Inventors: Chunwang DONG, Jinan (CN); Dandan QI, Jinan (CN); Zhiwei CHEN, Jinan (CN); Yali SHI, Jinan (CN); Mengqi GUO, Jinan (CN); Min LU, Jinan (CN); Fengzhi AN, Jinan (CN); Jianhua GAO, Jinan (CN); Qijun YUAN, Jinan (CN); Yusheng LI, Jinan (CN); Fan'an ZHANG, Jinan (CN); Yue ZHAO, Jinan (CN); Dewen WANG, Jinan (CN)

(73) Assignees: Shandong Academy of Agricultural Sciences, Jinan (CN); Rizhao City Lanshan District New Rural Construction and Development Co., Ltd., Rizhao (CN)

(21) Appl. No.: 19/251,890

(22) Filed: Jun. 27, 2025

(30) Foreign Application Priority Data
Jun. 28, 2024 (CN) 202410854078.3

(51) Int. Cl. G01N 33/02; G16C 20/70

(52) U.S. Cl. CPC G01N 33/025; G16C 20/70

(57) ABSTRACT

A method for identifying a producing area of Rizhao green tea is provided to identify Rizhao green tea in a trace amount accurately, conveniently, and rapidly, providing a new theoretical method and scientific basis for tracing the producing area of the Rizhao green tea. The method includes the following steps: S1, collection and preprocessing of tea leaf samples; S2, thermogravimetric analysis-Fourier transform infrared spectroscopy (TGA-FTIR) coupled testing; S3, infrared spectrogram analysis; S4, thermogravimetric-differential thermogravimetric spectrogram analysis; S5, estimation of kinetic parameters of the Rizhao green tea; S6, calculation of the pyrolysis parameters of the Rizhao green tea; and S7, establishment of an Ea prediction model for Rizhao green tea. A prediction model is established for the activation energy Ea of a Rizhao green tea leaf sample Y1, and an input to the prediction model is merely three characteristic parameters. The problem of model complexity is fundamentally solved.

### CROSS REFERENCE TO THE RELATED APPLICATIONS

This application is based upon and claims priority to Chinese Patent Application No. 202410854078.3, filed on Jun. 28, 2024, the entire contents of which are incorporated herein by reference.

### TECHNICAL FIELD

The present disclosure relates to the field of Rizhao green tea identification technology, and in particular, to a method for identifying a producing area of Rizhao green tea.

### BACKGROUND

Rizhao green tea, which is produced under advantaged natural conditions and is of long standing, has become one of the globally well-known green teas. Rizhao green tea possesses rich tastes and fragrances and can be used for treating many diseases. How to identify the producing area of Rizhao green tea is conducive to fighting against counterfeit and shoddy products, protecting consumers' rights and interests, and stabilizing the market. The traditional identification methods generally include a sensory evaluation method and a chemical method. The sensory evaluation method will be greatly affected by human factors and external factors, may require a lot of manpower and material resources, and is costly and time-consuming. The chemical method includes cumbersome steps and is costly, and requires the use of a large amount of organic reagents, leading to great environmental hazards. More importantly, the contents of some constituents in Rizhao green tea can be used as indicators for quality inspection, but are insufficient to become the necessary and sufficient conditions for identifying a producing area. Therefore, there is a need to establish an accurate, reliable, rapid, and simple identification method. Biomass pyrolysis is a quantitative analysis technique in which the quality change of a sample can be monitored when a furnace temperature rises from room temperature to thousands of degrees centigrade under a stable or changing gas flow. Thermogravimetric analysis (TGA) is coupled with the Fourier transform infrared spectroscopy (FTIR) for use in detecting the gas released in the biomass pyrolysis process. This combination incorporates the quantitative capability of the thermogravimetry (TG) and the identification capability of the FTIR. TGA-FTIR has the characteristics of simple operation, no solvent, a small number of samples, rapid detection, and straightforward spectra and thus has been widely applied.

### SUMMARY

An objective of the present disclosure is to provide a method for identifying a producing area of Rizhao green tea for overcoming the defects of high cost, complex operation procedures, and poor stability of an existing method for tracing a producing area of Rizhao green tea and for identifying Rizhao green tea in a trace amount accurately, conveniently, and rapidly, providing a new theoretical method and scientific basis for tracing the producing area of the Rizhao green tea.

The technical solution adopted to solve the technical problems of the present disclosure is as follows: a method for identifying a producing area of Rizhao green tea includes the following steps:
S1, collection and preprocessing of tea leaf samples:
collecting a Rizhao green tea leaf sample Y1, a tea leaf sample Y2 from a first province, a tea leaf sample Y3 from a second province, and a tea leaf sample Y4 from a third province, and preliminarily grinding the tea leaf samples, where the tea leaf samples are curled green teas;
S2, thermogravimetric analysis-Fourier transform infrared spectroscopy (TGA-FTIR) coupled testing:
performing the TGA-FTIR coupled testing using thermal analyzer STA449 from NETZSCH or thermal analyzer Nicolet 1s10 from Thermo Fisher Scientific; before testing, levelling a balance of the thermal analyzer, weighing 10 mg±0.5 mg test sample in a weighing area of the balance, and then turning on the thermal analyzer for testing;
S3, infrared spectrogram analysis:
plotting three-dimensional spectrograms of smokes generated by the four tea leaf samples in a pyrolysis process, and two-dimensional spectrograms at maximum decomposition rates in the pyrolysis process;
S4, thermogravimetric-differential thermogravimetric (TG-DTG) spectrogram analysis:
processing thermogravimetric data obtained from the TGA-FTIR coupled testing, importing temperatures and the thermogravimetric data in Origin software, performing first derivation on the thermogravimetric data to obtain differential thermogravimetric data, and plotting a TG-DTG curve spectrogram of the tea leaf samples with the temperatures as a horizontal axis and the thermogravimetric data and the processed differential thermogravimetric data as a vertical axis;
S5, estimation of kinetic parameters of the Rizhao green tea:
estimating activation energy using three integration methods, namely Kissinger method, Ozawa method, and Starink method:
(1) the Kissinger method:
a calculation equation of the Kissinger method being as follows:

$$ln ( \frac{β}{{T}^{2}} ) = ln ( \frac{A R}{E} ) - \frac{E}{R T} \qquad ( 1 )$$

where β represents a temperature rise rate, in units of K/min; T represents a characteristic temperature, in units of K; E represents the activation energy, in units of KJ/mol; R represents a Boltzmann gas constant, with a value of 8.314, in units of J/(mol· K); A represents a pre-exponential factor; and at different temperature rise rates, E is determined from a linear curve slope of

$$ln ( \frac{β}{{T}^{2}} ) to - \frac{1}{T} ;$$

(2) the Ozawa method:
a calculation equation of the Ozawa method being as follows:

$$ln ( β ) = ln ( \frac{A E a}{R g ( α )} ) - 5.331 - 1.052 \frac{E a}{R T} \qquad ( 2 )$$

where at different temperature rise rates, Ea, the activation energy, is determined by a linear curve slope of ln(β) to 1/T;
(3) the Starink method:
a calculation equation of the Starink method being as follows:

$$ln ( \frac{β}{{T}^{1.92}} ) = l g ( \frac{A R}{R g ( α )} ) - 0.312 - 1.0008 \frac{E a}{R T} \qquad ( 3 )$$

where the pre-exponential factor A is determined by the Kissinger method, and expressed as:

$$A = \frac{β Ea exp ( \frac{E a}{R T} )}{R {T}^{2}} \qquad ( 4 )$$

estimating three key thermodynamic parameters, namely enthalpy change ΔH, gibbs free energy change ΔG, and entropy change ΔS, by the following equations:

$$Δ H = E a - R {T}_{m} \qquad ( 5 )$$

$$Δ G = E a + R {T}_{m} ln ( \frac{{k}_{b} {T}_{m}}{h A} ) \qquad ( 6 )$$

$$Δ S = ( Δ G - Δ H ) / {T}_{m} \qquad ( 7 )$$

where k_{b} represents the Boltzmann constant, the value of k_{b} being 1.38×10⁻²³ J/K; h represents the Planck constant, the value of h being 6.626×10⁻³⁴ J·s;
α represents a weight loss fraction or a thermal conversion rate, a calculation equation of which is as follows:

$$α = ( {m}_{0} - m ) / ( {m}_{0} - {m}_{f} ) \qquad ( 8 )$$

where m₀ represents an initial mass of a sample, m represents a mass of a pyrolyzed sample at a given moment, and mf represents a final residue mass after thermal decomposition;
when the temperature rise rate β is constant,

$$β = \frac{d α}{d T} = \frac{d α}{d t} \frac{d t}{d T} \qquad ( 9 )$$

obtaining basic data of pyrolysis of the four tea leaf samples at different conversion rates α (a value range of α being 0.1 to 0.9, with an increment of 0.1), performing linear fitting by the Kissinger, Ozawa, and Starink methods at different heating rates, respectively, and at each conversion rate α, calculating corresponding activation energy and pre-exponential factor and plotting a point plot;
S6, calculation of the pyrolysis parameters of the Rizhao green tea
calculating the thermodynamic parameters enthalpy change ΔH, gibbs free energy change ΔG, and entropy change ΔS at each conversion rate; and
S7, establishment of an activation energy Ea prediction model for Rizhao green tea
calculating an activation energy Ea value of the Rizhao green tea leaf sample Y1 at each conversion rate α, establishing linear partial least squares regression (PLSR) and nonlinear support vector regression (SVR) and random forest (RF) regression prediction models with three different physical parameters, namely different conversion rates α and different temperatures T and temperature rise rates β corresponding to the different conversion rates α, as inputs to the models, and selecting an optimal Ea prediction model.

Further, step S1 may include: sieving the ground tea leaf samples through a 150-mesh screen, and selecting tea leaf particles below the 150-mesh screen as final test samples.

Further, step S2 may include: before turning on experimental apparatuses, setting basic parameters of a thermogravimetry-Fourier transform infrared spectrometer, setting a test atmosphere to a high-purity nitrogen atmosphere, setting a flow velocity to 60 mL/min, setting a heating temperature range of thermogravimetric analysis to be from room temperature to 800° C., and setting the temperature rise rate β to 10° C./min, 20° C./min, and 30° C./min at the thermogravimetric analysis.

Further, step S4 may include: performing signal smoothing on the differential thermogravimetric data, selecting an adjacent average method, and setting a number of window points to 20, thereby obtaining the processed differential thermogravimetric data.

Further, the pyrolysis process of the Rizhao green tea may be mainly divided into three stages:
at a first stage, the temperature is within a range from room temperature to 192° C., and the weight of the Rizhao green tea decreases slowly with a mass loss of about 4%, which mainly results from loss of free moisture in the tea leaf caused by evaporation;
at a second stage, the weight loss of the tea leaf is maximum; an active pyrolysis zone is created; a temperature range of the second stage is from 192° C. to 501° C.; the mass loss of the Rizhao green tea is 55.4%; and in the interval, fragrant constituents (e.g., micromolecular alcohols and ethers) in the tea leaf are decomposed and volatilized, and lignin, cellulose, and hemicellulose undergo violent pyrolytic reaction due to temperature rise, and are gradually carbonized, generating a large amount of gas; and
at a third stage, which is a carbonization stage within a range above 501° C., ash and charcoal are left as a residue is decomposed slowly at the stage; inorganic ash is volatilized and decomposed; the pyrolysis of the lignin occurs within a wide temperature range of 192° C. to 800° C.; a DTG curve after 501° C. is results of lignin decomposition, and only slight mass loss occurs in the range of the DTG curve.

Further, step S7 may include: before modeling, unifying, using four different preprocessing techniques, different conversion rates α, temperatures T, and temperature rise rates β to a same standard so as to optimize prediction models.

Further, in step S7, the four different preprocessing techniques may be standard normal variate (SNV) preprocessing, multiplicative scatter correction (MSC), Medfilt, and normalization and standardization (Z-score).

Further, a prediction model having maximum precision may be selected according to a value of Rc, a value of Rp, and a value of RPD as the random forest RF regression prediction model established through SNV preprocessing.

The present disclosure has the beneficial effects: (1) the present disclosure specifies the thermogravimetric curve spectrograms and the differential thermogravimetric curve spectrograms of the tea leaf samples of different producing areas, and results indicate that there are obvious differences in combustion characteristics in the combustion process between the Rizhao green tea leaf sample Y1 and the green teas of other producing areas. Compared with the other three tea leaf samples, the Rizhao green tea leaf sample Y1 has the characteristics of high initial temperature, slow decomposition, low cut-off temperature, high decomposition speed at the major thermal decomposition stage. (2) The three-dimensional spectrograms of smokes generated by the tea leaf samples of different producing areas in the thermogravimetric process are specified, and results indicate that the Rizhao green tea leaf sample Y1 has obvious differences in the absorption peak and the absorption peak size of the three-dimensional spectrogram from other tea leaf samples, and has higher CO₂ intensity than other tea leaf samples in the range from 2229 cm⁻¹ to 2404 cm⁻¹. The generation of SO₂ is detected within the range of 1383 cm⁻¹, which is due to the decomposition of sulfur-containing organic and inorganic compounds in the samples. Other absorption peaks represent HCN (674 cm⁻¹), NO (1741 cm⁻¹), C═C (1440 cm⁻¹ to 1587 cm⁻¹), CO (2185 cm⁻¹), and HCl (2935 cm⁻¹), respectively, and these chemical substances are major gaseous products in the combustion process. (3) The kinetic parameters and the thermodynamic parameters of the Rizhao green tea leaf sample Y1 and other tea leaf samples are specified. At all the stages of the pyrolytic reaction, the Rizhao green tea leaf sample Y1 has higher Ea than the tea leaf sample Y3 and the tea leaf sample Y4. Within the range 0.1<α<0.5, the Rizhao green tea leaf sample Y1 has higher Ea than the tea leaf sample Y2. Within the range 0.6<α<0.8, the Rizhao green tea leaf sample Y1 has lower Ea than the tea leaf sample Y2. At the later stage of the reaction, the Rizhao green tea leaf sample Y1 has higher Ea than the tea leaf sample Y2. This shows that the whole pyrolysis process exhibits an extremely complex reaction mechanism. A prediction model is established for the activation energy Ea of a Rizhao green tea leaf sample Y1, and an input to the prediction model is merely three characteristic parameters. The problem of model complexity is fundamentally solved. The established regression models have high prediction precision and can effectively predict the activation energy EA value of an unknown Rizhao green tea leaf sample and identify the producing area thereof.

### BRIEF DESCRIPTION OF THE DRAWINGS

FIG. 1A is a three-dimensional spectrogram of smoke generated by a tea leaf sample Y1 in the pyrolysis process;

FIG. 1B is a three-dimensional spectrogram of smoke generated by a tea leaf sample Y2 in the pyrolysis process;

FIG. 1C is a three-dimensional spectrogram of smoke generated by a tea leaf sample Y3 in the pyrolysis process;

FIG. 1D is a three-dimensional spectrogram of smoke generated by a tea leaf sample Y4 in the pyrolysis process;

FIG. 2 is diagram showing two-dimensional spectrograms of tea leaf samples Y1-Y4 at maximum decomposition rates in the pyrolysis process;

FIG. 3A is a diagram showing thermal degradation TG curves of the tea leaf sample Y1 at temperature rise rates of 10° C./min, 20° C./min, and 30° C./min;

FIG. 3B is a diagram showing thermal degradation DTG curves of the tea leaf sample Y1 at the temperature rise rates of 10° C./min, 20° C./min, and 30° C./min;

FIG. 3C is a diagram showing thermal degradation TG curves of the tea leaf sample Y2 at the temperature rise rates of 10° C./min, 20° C./min, and 30° C./min;

FIG. 3D is a diagram showing thermal degradation DTG curves of the tea leaf sample Y2 at the temperature rise rates of 10° C./min, 20° C./min, and 30° C./min;

FIG. 3E is a diagram showing thermal degradation TG curves of the tea leaf sample Y3 at the temperature rise rates of 10° C./min, 20° C./min, and 30° C./min;

FIG. 3F is a diagram showing thermal degradation DTG curves of the tea leaf sample Y3 at the temperature rise rates of 10° C./min, 20° C./min, and 30° C./min;

FIG. 3G is a diagram showing thermal degradation TG curves of the tea leaf sample Y4 at the temperature rise rates of 10° C./min, 20° C./min, and 30° C./min;

FIG. 3H is a diagram showing thermal degradation DTG curves of the tea leaf sample Y4 at the temperature rise rates of 10° C./min, 20° C./min, and 30° C./min;

FIG. 4A is a diagram showing regression curves of conversion points (α) at 3 temperature rise rates plotted for the tea leaf sample Y1 using Kissinger method;

FIG. 4B is a diagram showing regression curves of conversion points (α) at 3 temperature rise rates plotted for the tea leaf sample Y1 using Ozawa method;

FIG. 4C is a diagram showing regression curves of conversion points (α) at 3 temperature rise rates plotted for the tea leaf sample Y1 using Starink method;

FIG. 4D is a diagram showing regression curves of conversion points (α) at 3 temperature rise rates plotted for the tea leaf sample Y2 using the Kissinger method;

FIG. 4E is a diagram showing regression curves of conversion points (α) at 3 temperature rise rates plotted for the tea leaf sample Y2 using the Ozawa method;

FIG. 4F is a diagram showing regression curves of conversion points (α) at 3 temperature rise rates plotted for the tea leaf sample Y2 using the Starink method;

FIG. 4G is a diagram showing regression curves of conversion points (α) at 3 temperature rise rates plotted for the tea leaf sample Y3 using the Kissinger method;

FIG. 4H is a diagram showing regression curves of conversion points (α) at 3 temperature rise rates plotted for the tea leaf sample Y3 using the Ozawa method;

FIG. 4I is a diagram showing regression curves of conversion points (α) at 3 temperature rise rates plotted for the tea leaf sample Y3 using the Starink method;

FIG. 4J is a diagram showing regression curves of conversion points (α) at 3 temperature rise rates plotted for the tea leaf sample Y4 using the Kissinger method;

FIG. 4K is a diagram showing regression curves of conversion points (α) at 3 temperature rise rates plotted for the tea leaf sample Y4 using the Ozawa method;

FIG. 4L is a diagram showing regression curves of conversion points (α) at 3 temperature rise rates plotted for the tea leaf sample Y4 using the Starink method;

FIG. 5 is an analysis chart of Ea values (a) of four tea leaf samples at each conversion point using the Kissinger, Ozawa, and Starink methods;

FIG. 6A is a diagram showing values of a thermodynamic parameter enthalpy change ΔH of the tea leaf sample Y1 at different conversion rates;

FIG. 6B is a diagram showing values of a thermodynamic parameter gibbs free energy ΔG of the tea leaf sample Y1 at different conversion rates;

FIG. 6C is a diagram showing values of a thermodynamic parameter entropy change ΔS of the tea leaf sample Y1 at different conversion rates;

FIG. 6D is a diagram showing values of the thermodynamic parameter enthalpy change ΔH of the tea leaf sample Y2 at different conversion rates;

FIG. 6E is a diagram showing values of the thermodynamic parameter gibbs free energy ΔG of the tea leaf sample Y2 at different conversion rates;

FIG. 6F is a diagram showing values of the thermodynamic parameter entropy change ΔS of the tea leaf sample Y2 at different conversion rates;

FIG. 6G is a diagram showing values of the thermodynamic parameter enthalpy change ΔH of the tea leaf sample Y3 at different conversion rates;

FIG. 6H is a diagram showing values of the thermodynamic parameter gibbs free energy ΔG of the tea leaf sample Y3 at different conversion rates;

FIG. 6I is a diagram showing values of the thermodynamic parameter entropy change ΔS of the tea leaf sample Y3 at different conversion rates;

FIG. 6J is a diagram showing values of the thermodynamic parameter enthalpy change ΔH of the tea leaf sample Y4 at different conversion rates;

FIG. 6K is a diagram showing values of the thermodynamic parameter gibbs free energy ΔG of the tea leaf sample Y4 at different conversion rates;

FIG. 6L is a diagram showing values of the thermodynamic parameter entropy change ΔS of the tea leaf sample Y4 at different conversion rates;

FIG. 7 is a scatter diagram showing measured values and prediction values of an RF prediction model after SNV preprocessing;

FIG. 8 is a diagram showing values of Ea, R², and A calculated by the Kissinger method for four tea leaf samples;

FIG. 9 is a diagram showing values of Ea, R², and A calculated by the Ozawa method for four tea leaf samples;

FIG. 10 is a diagram showing values of Ea, R², and A calculated by the Starink method for four tea leaf samples; and

FIG. 11 is a performance evaluation diagram of three prediction models by different preprocessing methods.

### DETAILED DESCRIPTION OF THE EMBODIMENTS

The present disclosure, which is based on the thermogravimetric analysis-Fourier transform infrared spectroscopy (TGA-FTIR) coupled technique, aimed at tracing the producing area of Rizhao green tea in combination with the machine learning technique. Curled green tea leaf samples of four different producing areas are collected. TGA-FTIR coupled testing is performed on the tea leaf samples of different producing areas, and differentiation is performed on the obtained thermogravimetric data to obtain differential thermogravimetric (DTG) data. The kinetic parameters and the thermodynamic parameters of the tea leaf samples of different producing areas are determined using three model-free methods Kissinger, Ozawa, and Starink, respectively. Finally, an activation energy Ea prediction model for Rizhao green tea is established for realizing the tracing of the producing area of the Rizhao green tea. Specific steps are as follows: S1, tea leaf samples are collected and preprocessed.

A Rizhao green tea leaf sample Y1, a tea leaf sample Y2 from a first province, a tea leaf sample Y3 from a second province, and a tea leaf sample Y4 from a third province are collected. These tea leaf samples are curled green teas. Before formal testing, all the tea leaf samples are stored in a low temperature environment at −5° C. Preliminarily, the tea leaf samples are ground manually, and a 150-mesh screen is provided. The ground tea leaf samples are sieved through the 150-mesh screen, and tea leaf particles below the 150-mesh screen are selected as final test samples. S2, TGA-FTIR coupled testing is performed.

The TGA-FTIR coupled testing is performed using thermal analyzer STA449 from NETZSCH or thermal analyzer Nicolet 1s10 from Thermo Fisher Scientific. Before testing, a balance of the thermal analyzer is levelled. The method of levelling is as follows: crucibles of equal mass are put on two ends of the balance of the thermal analyzer. After the balance is completely steady, a zero clearing operation is performed, guaranteeing the accuracy of the balance. 10 mg±0.5 mg test sample is weighed in a weighing area of the balance. Before the thermal analyzer is turned on, basic parameters of a thermogravimetry-Fourier transform infrared spectrometer are set. A test atmosphere is set to a high-purity nitrogen atmosphere, a flow velocity is set to 60 mL/min, a heating temperature range of thermogravimetric analysis is set to be from room temperature to 800° C., and a temperature rise rate β is set to 10° C./min, 20° C./min, and 30° C./min at the thermogravimetric analysis. After the above basic parameters are set, the thermal analyzer is turned on for testing.

S3, infrared spectrogram analysis is performed.

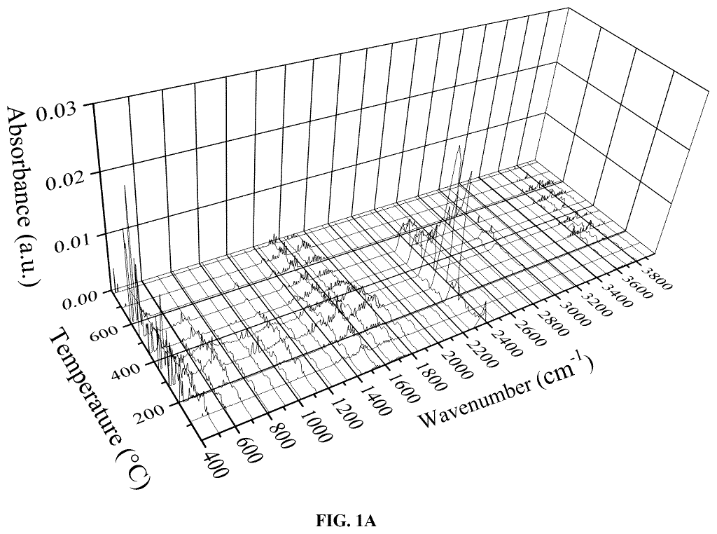

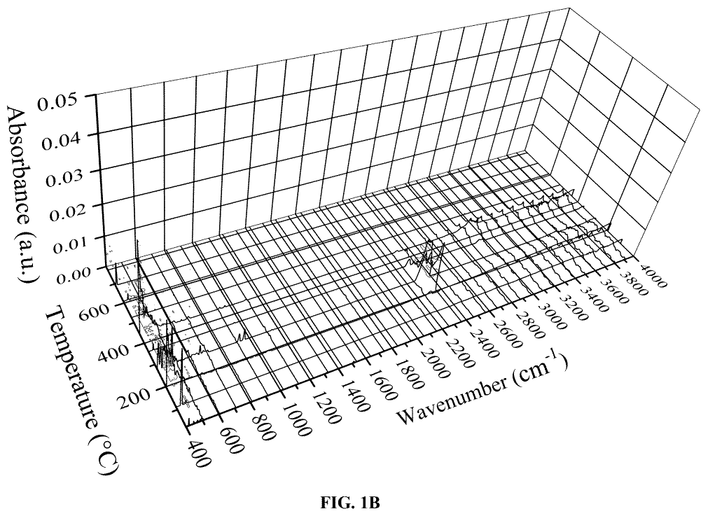

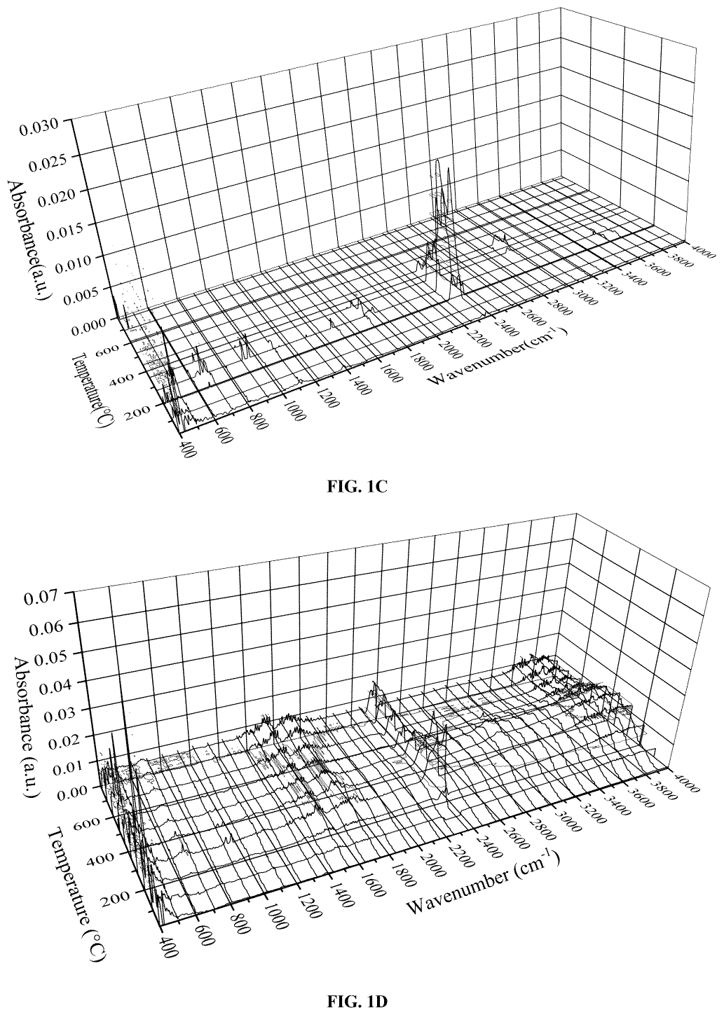

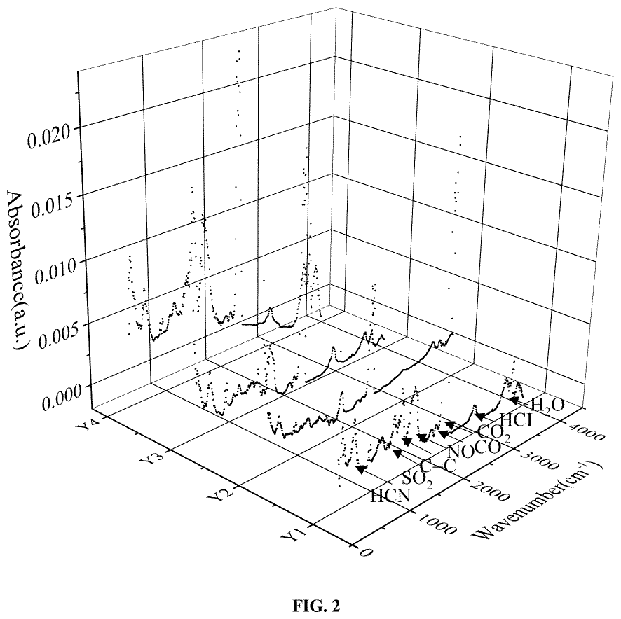

As shown in FIGS. 1A-1D, three-dimensional diagrams are plotted with infrared data. As shown in FIG. 2, two-dimensional diagrams are plotted with a spectrogram at a maximum decomposition rate. Part of infrared absorption peaks can be determined. The infrared spectrograms of the four tea leaf samples have the maximum absorbances in wavenumber ranges of 2229 cm⁻¹ to 2404 cm⁻¹ and 489 cm⁻¹ to 535 cm⁻¹, all corresponding to CO₂. The generation of CO₂ at a low temperature is mainly due to the decarboxylic reaction, e.g., decomposition of carboxyl and carbonyl. The generation of CO₂ at a high temperature is mainly caused by devolatilization of carbon-containing components and charcoal burning. The emission peak within the second range is more obvious than the emission peak within the first range, indicating that the emission of CO₂ is much intense within the higher temperature range in the combustion processes of the tea leaf samples from four different areas. Moreover, the Rizhao green tea leaf sample 1 has higher CO₂ intensity in the range of 2229 cm⁻¹ to 2404 cm⁻¹ than the other three tea leaf samples (the tea leaf sample Y2, the tea leaf sample Y3, and the tea leaf sample Y4), indicating that the emission of CO₂ in the combustion process of the Rizhao green tea is increasing. In the range of 3500 cm⁻¹ to 4000 cm⁻¹, there is H₂O generated. In the range of 1383 cm⁻¹, the generation of SO₂ is detected, which is due to the decomposition of sulfur-containing organic and inorganic compounds in the samples. Other absorption peaks represent HCN (674 cm⁻¹), NO (1741 cm⁻¹), C═C (1440 cm⁻¹ to 1587 cm⁻¹), CO (2185 cm⁻¹), and HCl (2935 cm⁻¹), respectively, and these chemical substances are major gaseous products in the combustion process. CO is mainly due to partial CO release from reaction with CO₂ residual coke (CO₂+C→2CO) and partial combustion of a carbon-containing substance (2C+O₂→2CO). The evolution of CO exhibits similar change features to CO₂, where the absorption intensity of CO₂ is 508 times that of CO, indicating that the combustible has been combusted completely.

S4, thermogravimetric-differential thermogravimetric (TG-DTG) spectrogram analysis is performed.

The thermogravimetric data obtained from the TG-FTIR testing is processed. Temperatures and the thermogravimetric data are imported in Origin software. First derivation is performed on the thermogravimetric data using the differentiation operation in mathematics to obtain differential thermogravimetric (DTG) data. Due to the existence of a noise interference factor in the experimental apparatuses, signal smoothing is performed on the DTG data. An adjacent average method is selected, and a number of window points is set to 20, thereby obtaining the processed DTG data. A TG-DTG curve spectrogram of the tea leaf samples of different producing areas is plotted with the temperatures as a horizontal axis and the thermogravimetric data and the processed DTG data as a vertical axis.

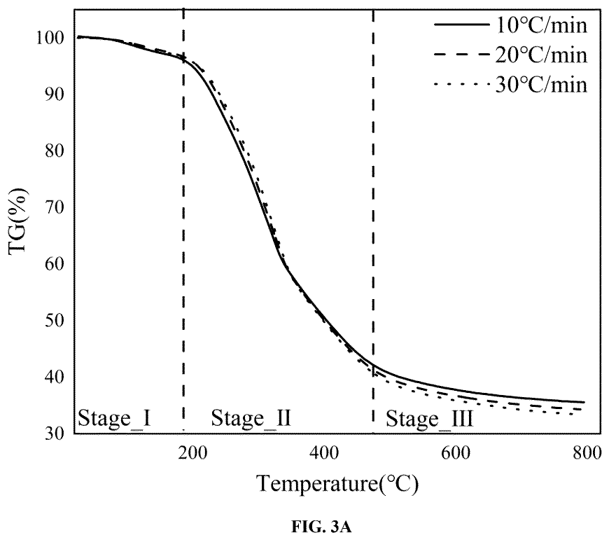

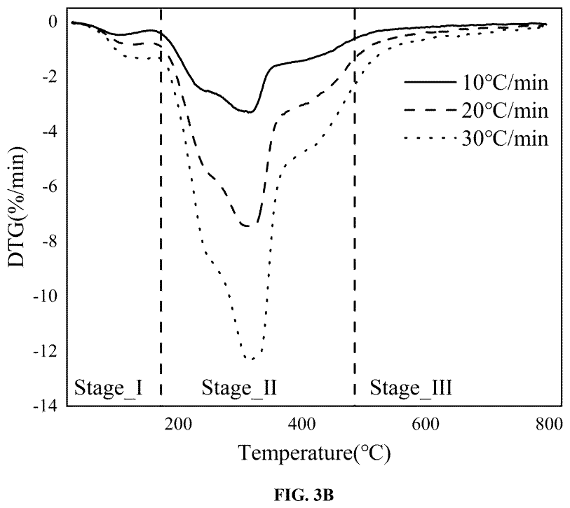

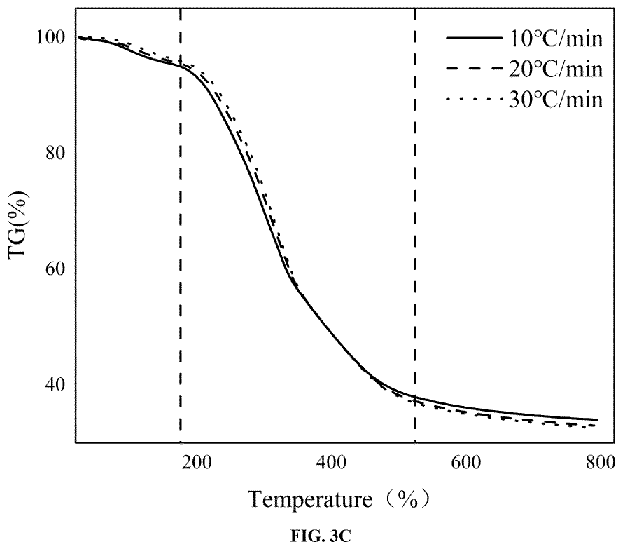

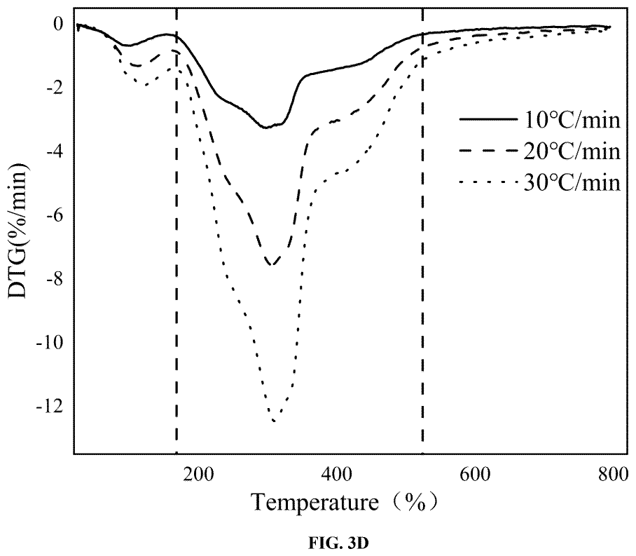

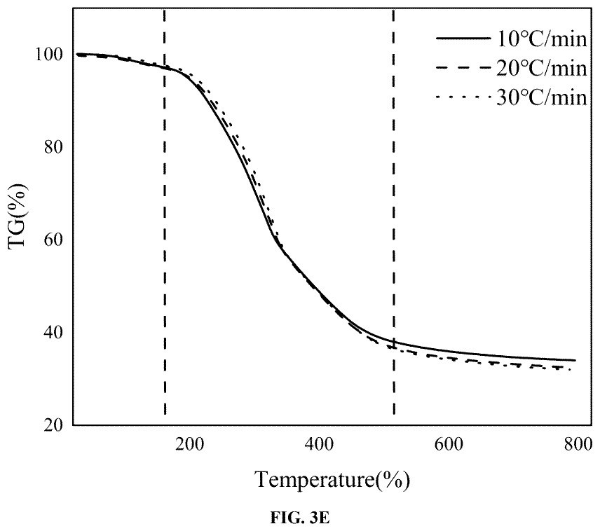

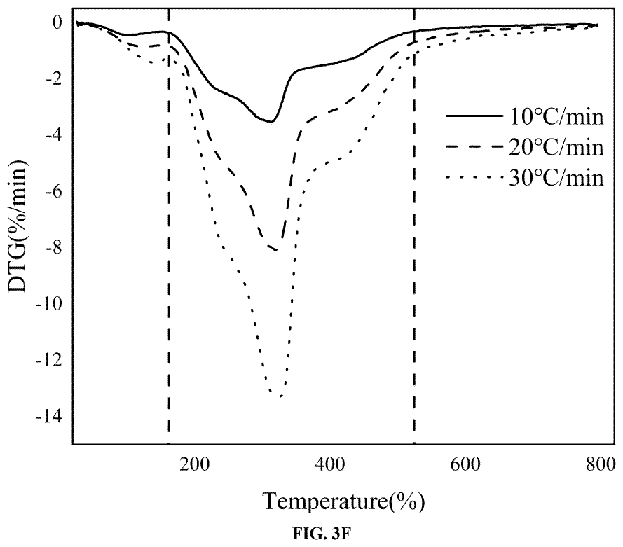

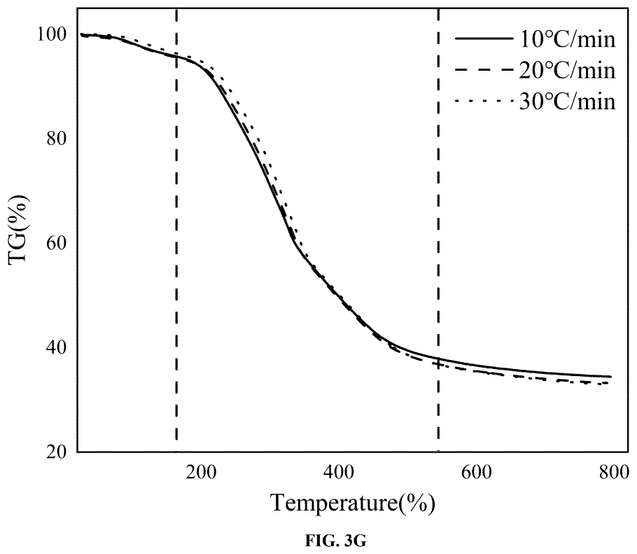

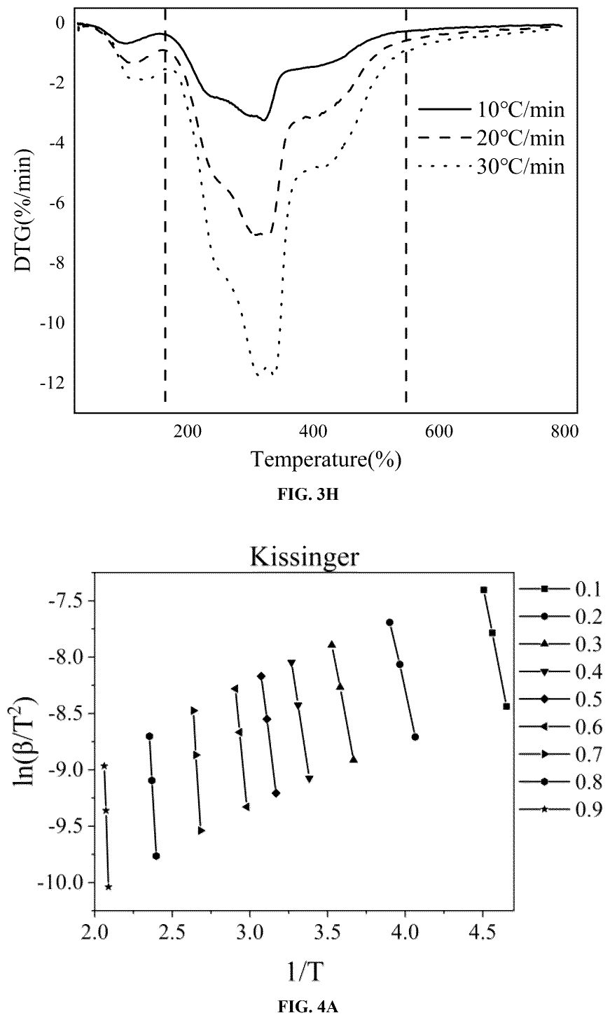

As can be seen from FIG. 3A and FIG. 3B, the pyrolysis process of the Rizhao green tea is mainly divided into three stages:
at a first stage, the temperature is within a range from room temperature to 192° C., and the weight of the Rizhao green tea decreases slowly with a mass loss of about 4%, which mainly results from loss of free moisture in the tea leaf caused by evaporation;
at a second stage, the weight loss of the tea leaf is maximum; an active pyrolysis zone is created; a temperature range of the second stage is from 192° C. to 501° C.; the mass loss of the Rizhao green tea is 55.4%; and in the interval, fragrant constituents (e.g., micromolecular alcohols and ethers) in the tea leaf are decomposed and volatilized, and lignin, cellulose, and hemicellulose undergo violent pyrolytic reaction due to temperature rise, and are gradually carbonized, generating a large amount of gas; and
at a third stage, which is a carbonization stage within a range above 501° C., ash and charcoal are left as a residue is decomposed slowly at the stage; inorganic ash is volatilized and decomposed; the pyrolysis of the lignin occurs within a wide temperature range of 192° C. to 800° C.; therefore, a DTG curve after 501° C. is results of lignin decomposition, and only slight mass loss occurs in the range of the DTG curve.

On the whole, the greater the temperature rise rate, the greater the mass loss at the end of pyrolysis, and the faster the mass loss at the major pyrolysis stage.

As shown in FIG. 3C and FIG. 3D, the temperature boundary points of the three pyrolysis stages of the tea leaf sample Y2 are 177° C. and 523° C., respectively. The temperature range at the major thermal decomposition stage is wider, and the mass decreases by 57.4%. At this stage, the mass loss of the tea leaf sample Y1 is 2% less than that of the tea leaf sample Y2.

As shown in FIG. 3E and FIG. 3F, the major thermal decomposition stage of the tea leaf sample Y3 is from 162° C. to 522° C. with a mass loss of about 60.5%. At this stage, the mass loss of the tea leaf sample Y1 is 5.1% less than that of the tea leaf sample Y3.

As shown in FIG. 3G and FIG. 3H, the major thermal decomposition stage of the tea leaf sample 4 is from 163° C. to 543° C. with a mass loss of about 58.7%. At this stage, the mass loss of the tea leaf sample 1 is 3.3% less than that of the tea leaf sample 4. To sum up, compared with the tea leaf samples Y2, Y3, and Y4, the Rizhao green tea leaf sample Y1 has the characteristics of high initial temperature, slow decomposition, low cut-off temperature, high decomposition speed at the major thermal decomposition stage.

S5, kinetic parameters of the Rizhao green tea are estimated.

The activation energy is estimated using three integration methods: Kissinger method, Ozawa method, and Starink method. Three different models are employed to ensure the accuracy of the thermogravimetric experimental data.

### (1) the Kissinger Method:

The Kissinger method can determine the kinetic parameters of a solid-state reaction without determining a reaction mechanism. The Kissinger method is based on the following equation:

$$ln ( \frac{β}{{T}^{2}} ) = ln ( \frac{A R}{E} ) - \frac{E}{R T} \qquad ( 1 )$$

where β represents a temperature rise rate, in units of K/min; T represents a characteristic temperature, in units of K; E represents the activation energy, in units of KJ/mol; R represents a Boltzmann gas constant, with a value of 8.314, in units of J/(mol·K); and A represents a pre-exponential factor.

At different temperature rise rates, E is determined from a linear curve slope of

$$ln ( \frac{β}{{T}^{2}} ) to - \frac{1}{T} .$$

### (2) the Ozawa Method:

Flynn-Wall-Ozawa method is a non-model fitting analysis method in which a conversion rate α is regarded as a constant value and the temperature rise rate β is merely related to the reaction temperature T, no reaction mechanism is involved, an error caused by the selection of a reaction mechanism function is avoided, and an Ea value is directly ascertained. The Ozawa method is based on the following equation:

$$ln ( β ) = ln ( \frac{A E a}{R g ( α )} ) - 5.331 - 1.052 \frac{E a}{R T} \qquad ( 2 )$$

where at different temperature rise rates, Ea, the activation energy, is determined by a linear curve slope of ln(β) to

### (3) the Starink Method:

The Starink method is based on the following equation:

$$ln ( \frac{β}{{T}^{1.92}} ) = lg ( \frac{AR}{Rg ( α )} ) - 0.312 - 1.0008 \frac{Ea}{RT} \qquad ( 3 )$$

where the pre-exponential factor A is determined by the Kissinger method, and expressed as:

$$A = \frac{β Ea exp ( \frac{Ea}{RT} )}{{RT}^{2}} \qquad ( 4 )$$

Three key thermodynamic parameters, namely enthalpy change ΔH, gibbs free energy change ΔG, and entropy change ΔS, are estimated by the following equations:

$$Δ H = Ea - {RT}_{m} \qquad ( 5 )$$

$$Δ G = Ea + {RT}_{m} ln ( \frac{{k}_{b} {T}_{m}}{hA} ) \qquad ( 6 )$$

$$Δ S = ( Δ G - Δ H ) / {T}_{m} \qquad ( 7 )$$

where k_{b} represents the Boltzmann constant, the value of k_{b} being 1.38×10⁻²³ J/K; and h represents the Planck constant, the value of h being 6.626×10⁻³⁴ J·s.

α represents a weight loss fraction or a thermal conversion rate, a calculation equation of which is as follows:

$$α = ( {m}_{0} - m ) / ( {m}_{0} - {m}_{F} ) \qquad ( 8 )$$

where m₀ represents an initial mass of a sample, m represents a mass of a pyrolyzed sample at a given moment, and mf represents a final residue mass after thermal decomposition. When the temperature rise rate β is constant, it may be expressed as:

$$β = \frac{d α}{dT} = \frac{d α}{dt} \frac{dt}{dT} \qquad ( 9 )$$

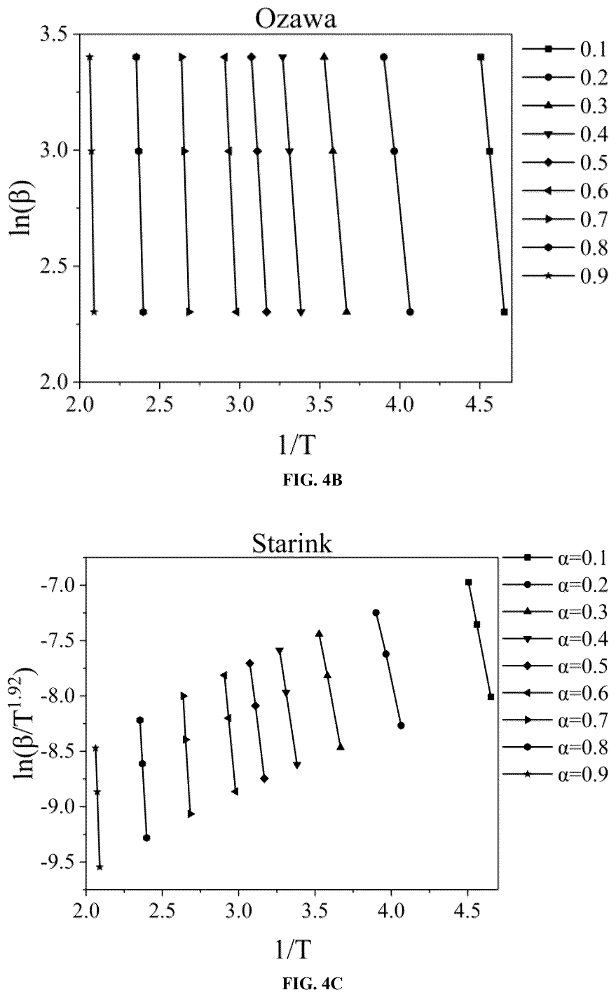

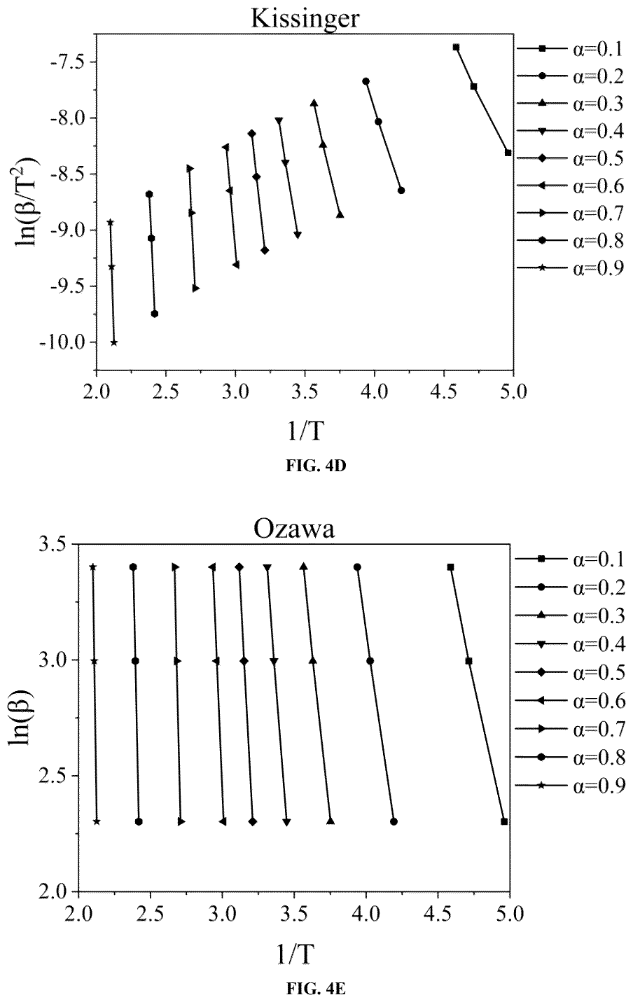

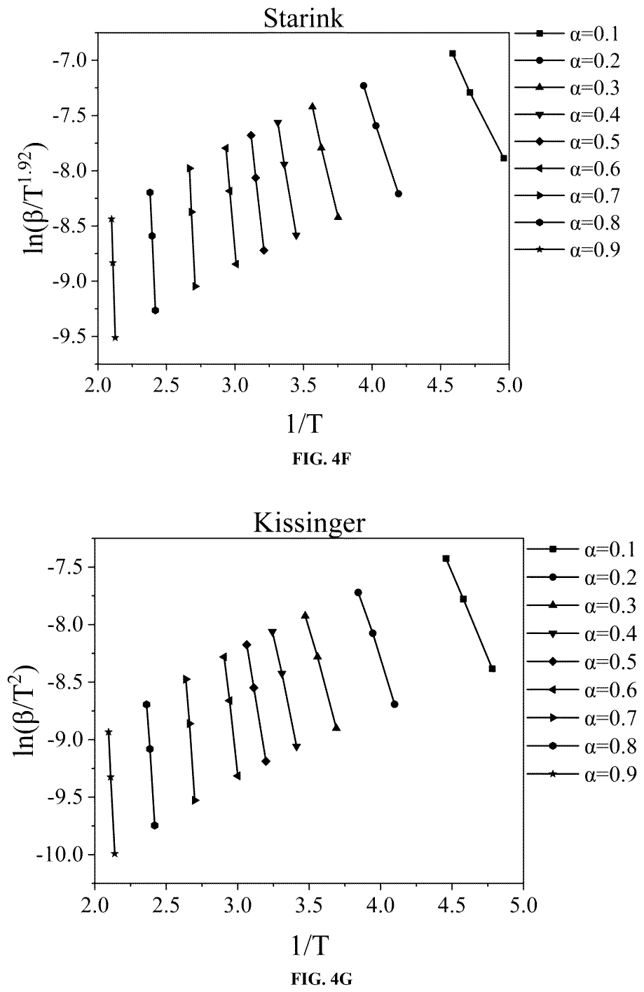

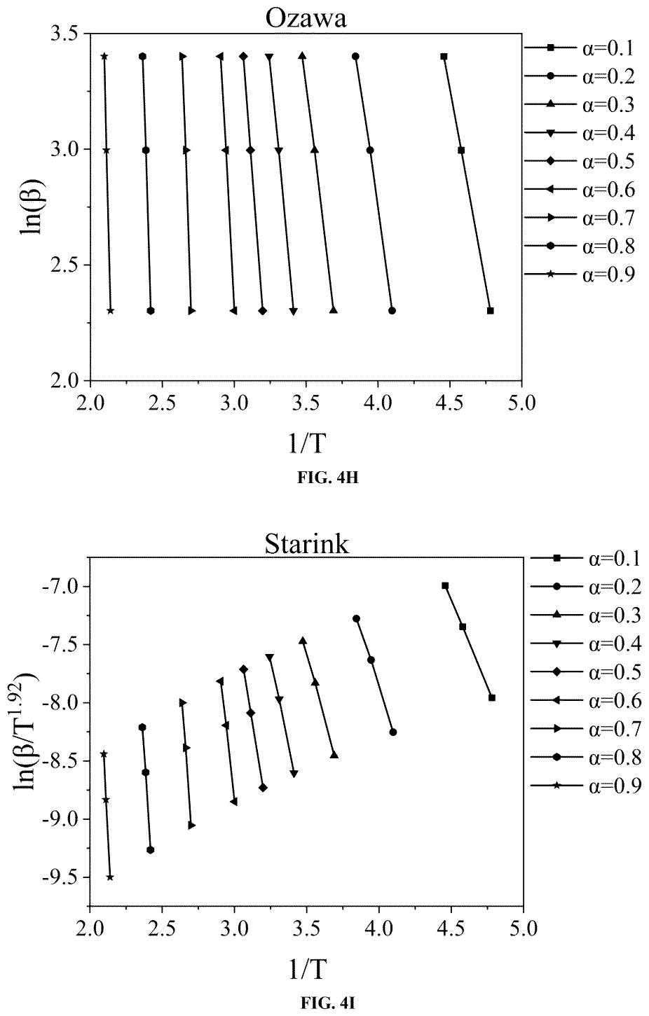

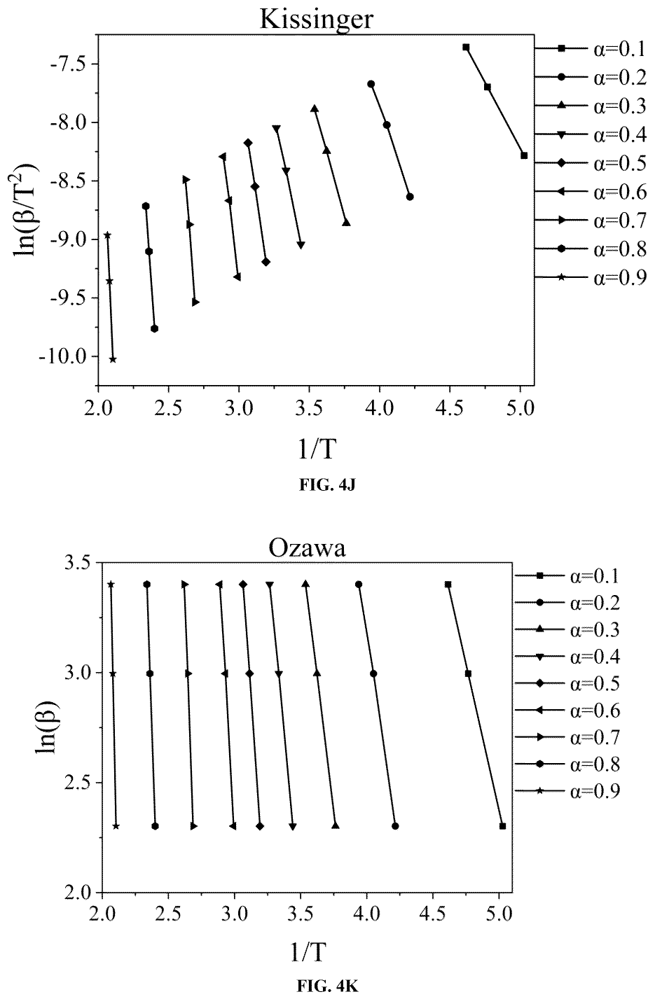

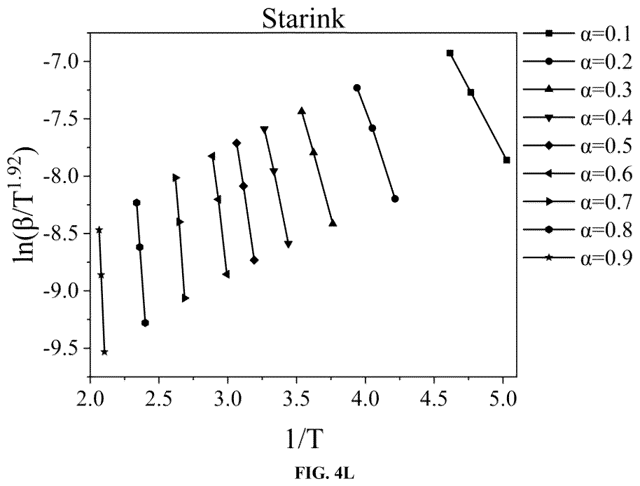

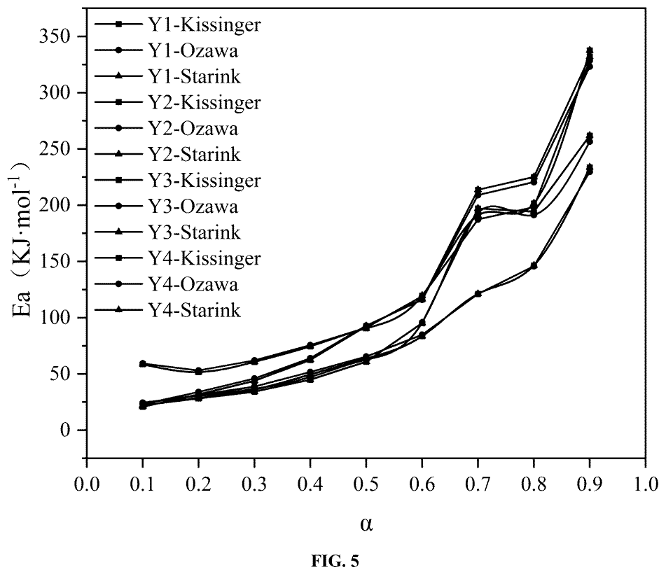

Basic data of pyrolysis of the tea leaf samples from four different areas are obtained at different conversion rates α (a value range of α being 0.1 to 0.9, with an increment of 0.1), and linear fitting is performed by the Kissinger, Ozawa, and Starink methods at three different heating rates, respectively. As shown in FIG. 4A to FIG. 4L, the X-axis is inverse (1/T, K−1) of the pyrolysis temperature, and the Y-axis is ln(β/T2)(K−1S−1), ln(β)(K S−1), and ln(β/T1.92)(K−1S−1). The linear fitting results at different a values are entirely satisfactory, indicating that the selected three integration methods can fully reflect the pyrolytic reactions of the tea leaf samples. At each conversion rate α, corresponding activation energy and pre-exponential factor are calculated and a point plot is plotted. For the Rizhao green tea leaf sample Y1, it can be seen from FIG. 5 that:

(1) The activation energy range calculated by the Kissinger method is from 58.696 KJ/mol to 337.602 KJ/mol, with an average value of 131.739 KJ/mol.

(2) The activation energy range calculated by the Ozawa method is from 59.245 KJ/mol to 328.528 KJ/mol, with an average value of 130.471 KJ/mol.

(3) The activation energy range calculated by the Starink method is from 58.794 KJ/mol to 337.653 KJ/mol, with an average value of 131.854 KJ/mol.

At all the stages of the pyrolytic reaction, the Rizhao green tea leaf sample Y1 has higher Ea than the tea leaf sample Y3 and the tea leaf sample Y4. Within the range 0.1<<<0.5, the Rizhao green tea leaf sample Y1 has higher Ea than the tea leaf sample Y2. Within the range 0.6<α<0.8, the Rizhao green tea leaf sample Y1 has lower Ea than the tea leaf sample Y2. At the later stage of the reaction, the Rizhao green tea leaf sample Y1 has higher Ea than the tea leaf sample Y2. This shows that the whole pyrolysis process exhibits an extremely complex reaction mechanism.

On the whole, the average activation energy of the Rizhao green tea leaf sample Y1 obtained based on the three integration methods is significantly higher than that of the other three tea leaf samples. Within the range 0.1<α<0.2, the Ea of the Rizhao green tea leaf sample Y1 exhibits a down trend from 58.696 KJ/mol to 51.644 KJ/mol. This interval is at the second stage of pyrolysis with the fastest mass loss, which is related to the strong combustion of fixed carbon, volatile components, and part of organic matter, and the decomposition of the cellulose. At the last stage of the reaction, by calculation using the Kissinger, Ozawa, and Starink methods, the Ea of the Rizhao green tea leaf sample Y1 rapidly increases from 201.585 KJ/mol to 337.602 KJ/mol, rapidly increases from 198.272 KJ/mol to 328.528 KJ/mol, and rapidly increases from 201.704 KJ/mol to 337.653 KJ/mol, respectively. This stage indicates that the lignin is decomposed and no active pyrolytic reaction occurs, and the product is charcoal.

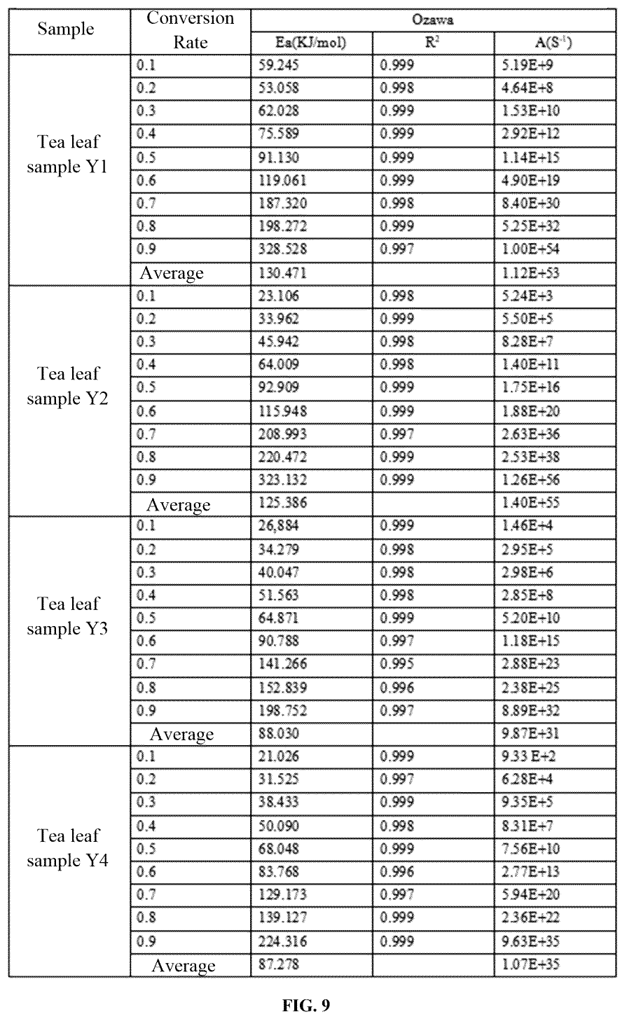

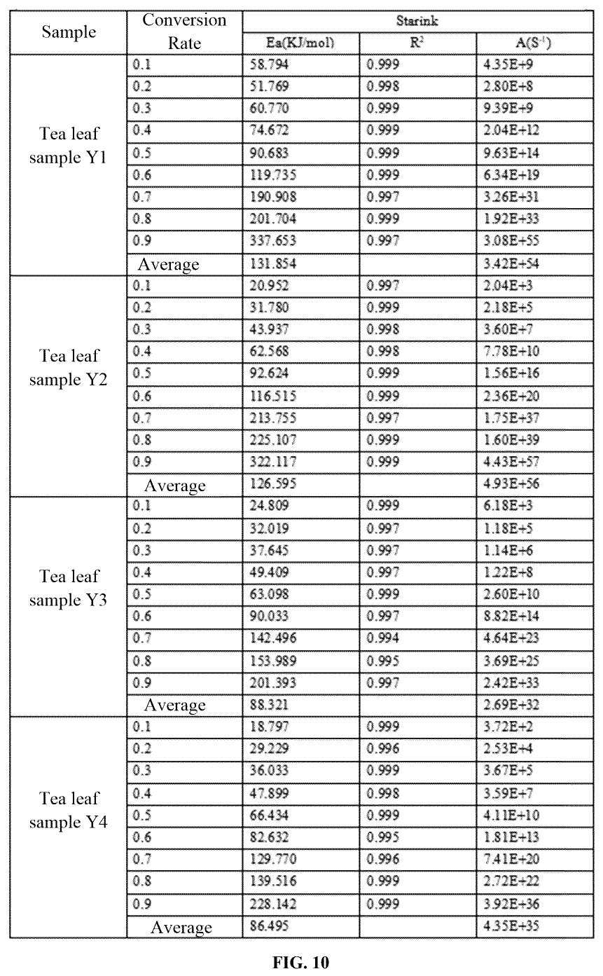

Under the condition of the temperature rise rate of 20° C./min, the pre-exponential factor A is estimated using the Kissinger, Ozawa, and Starink methods. In the whole pyrolysis process, with increasing conversion rate α, the value of the pre-exponential factor A of the Rizhao green tea leaf sample Y1 exponentially increases from 109 to 1054. The distribution range of the values of A is wide, indicating that the reaction occurring in the whole process is complex. The average value of the pre-exponential factor A of the Rizhao green tea leaf sample Y1 is slightly lower than that of the tea leaf sample Y2, and greater than those of the tea leaf sample Y3 and the tea leaf sample Y4. The greater the value of the pre-exponential factor A, the higher the reaction activity. As the temperature rises, for the tea leaf particles, the collision frequency between molecules is higher. The values of Ea, R², and the pre-exponential factor A of the four different tea leaf samples calculated by the 3 model-free methods are as shown in FIG. 8, FIG. 9, and FIG. 10.

S6, the pyrolysis parameters of the Rizhao green tea are calculated.

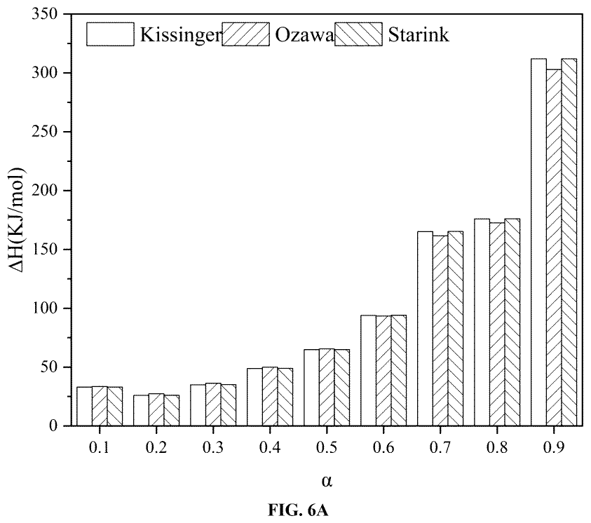

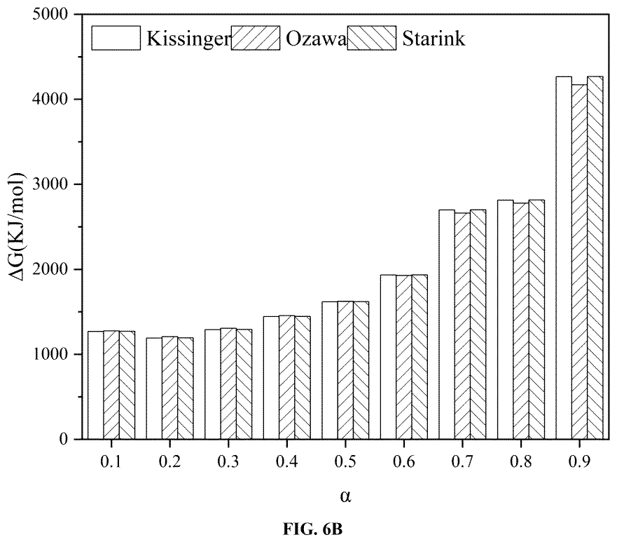

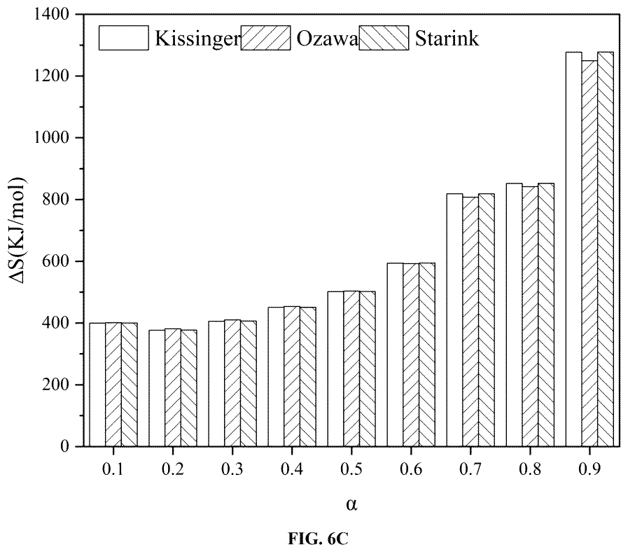

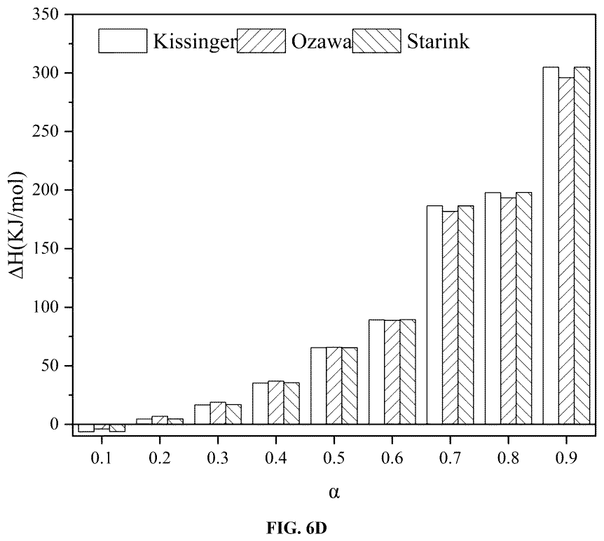

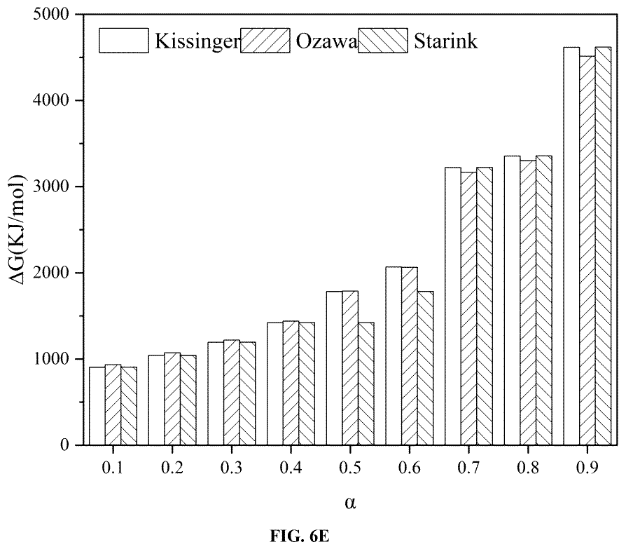

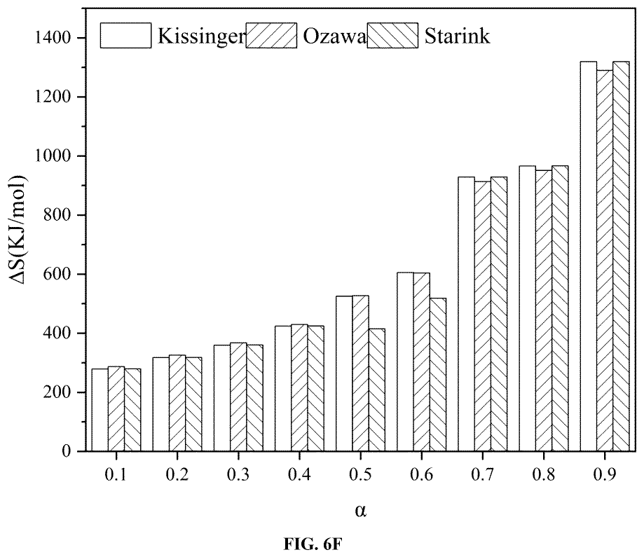

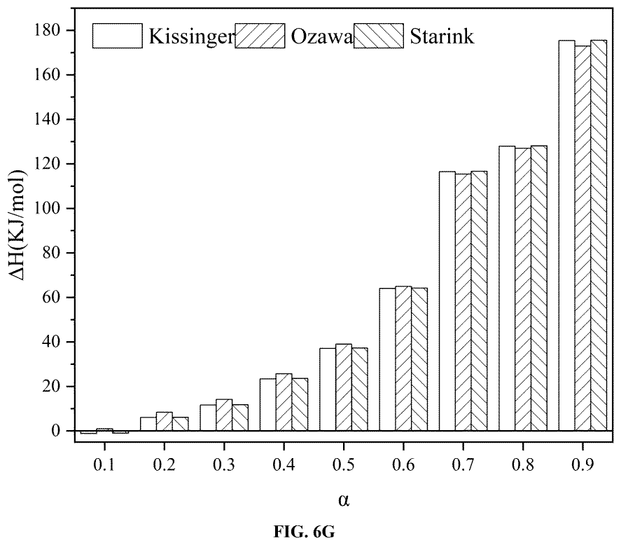

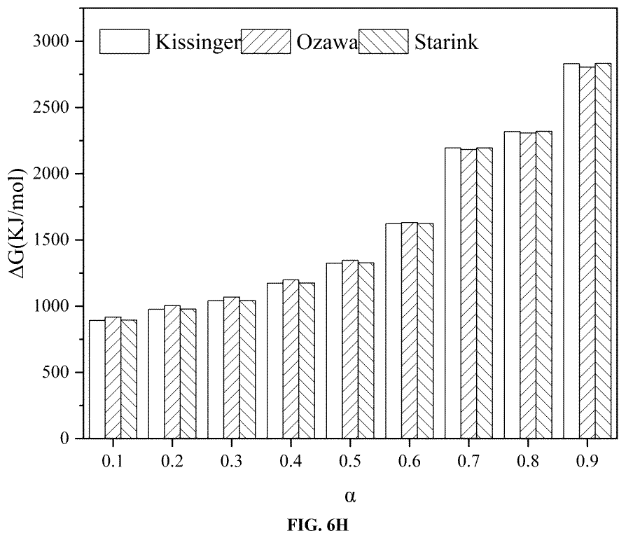

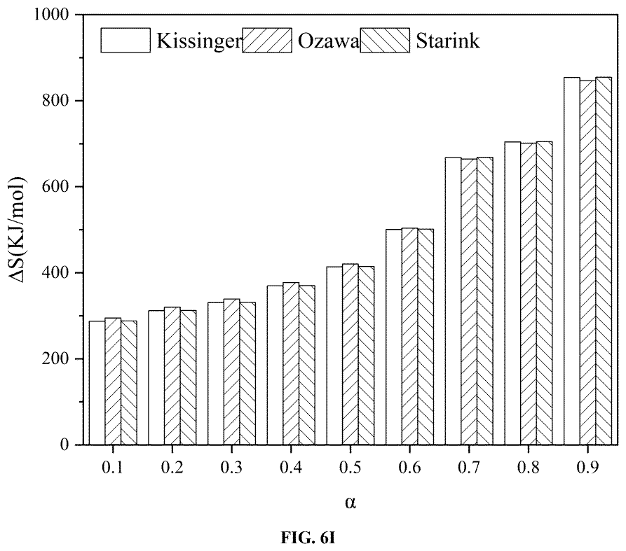

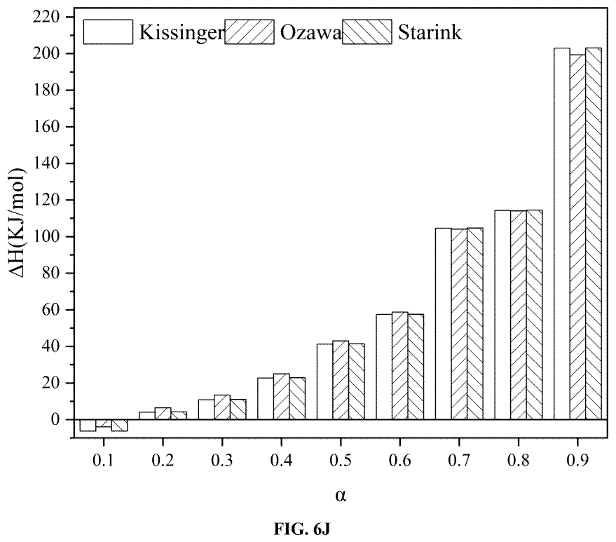

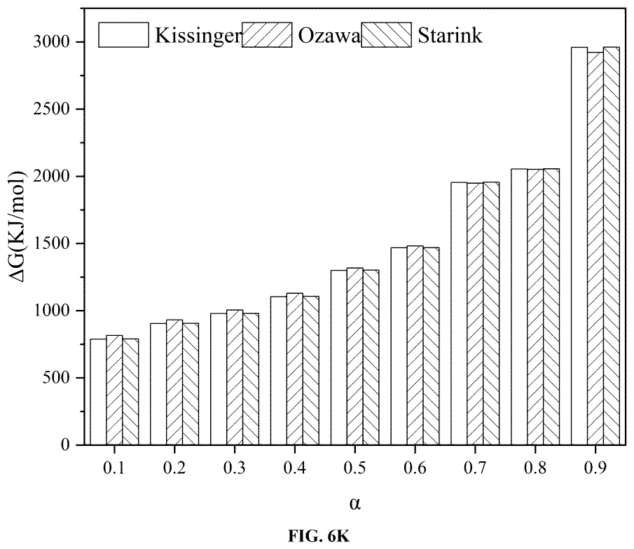

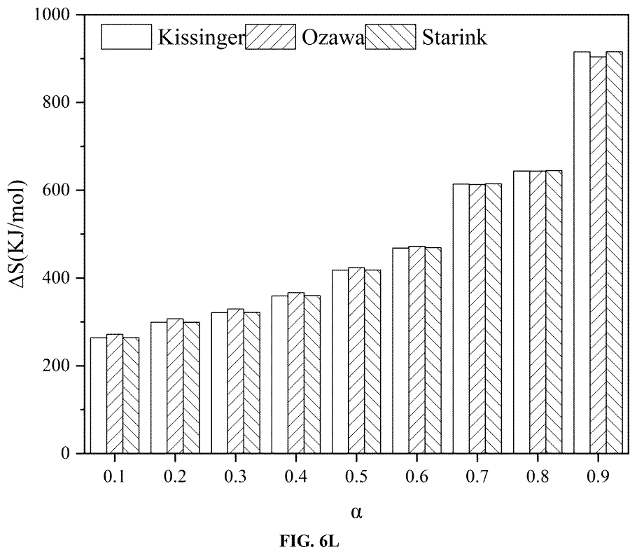

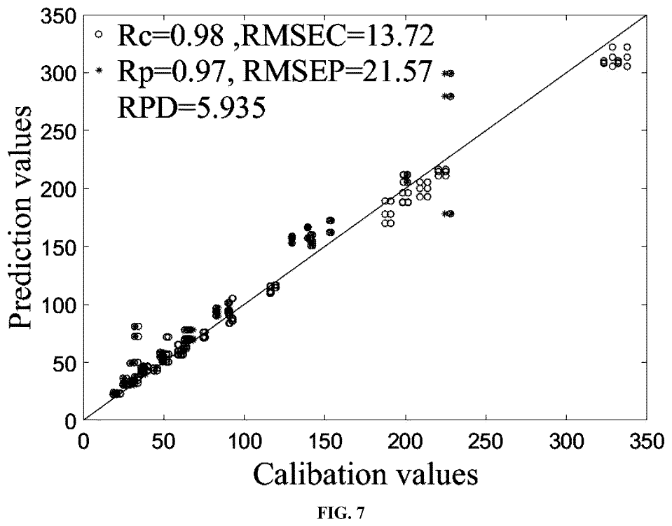

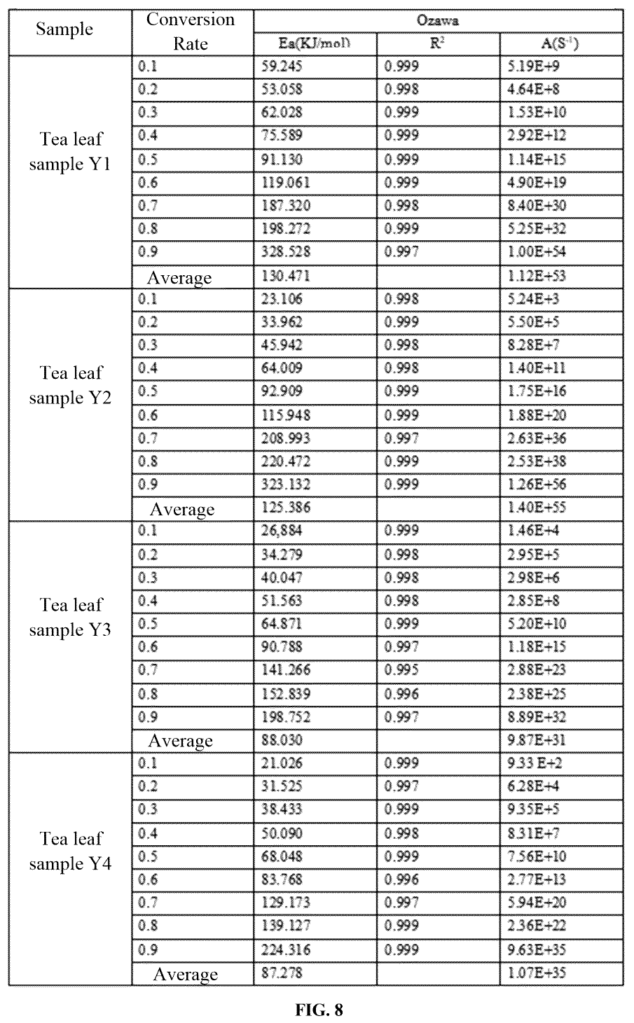

The thermodynamic parameters enthalpy change ΔH, gibbs free energy change ΔG, and entropy change ΔS at each conversion rate are calculated. As can be seen from FIG. 6A, FIG. 6B, FIG. 6C, and FIG. 7, the average values of αH of the Rizhao green tea leaf sample Y1 evaluated using the Kissinger, Ozawa, and Starink methods are 105.99 KJ/mol, 104.73 KJ/mol, and 106.11 KJ/mol, respectively. αH is greater than 0 in the whole process, indicating that heat is absorbed in the whole process. The difference between αH and Ea is about 20 KJ/mol. This small difference indicates that the conversion between them has a small energy barrier, which is conducive to the formation of the product. The enthalpy change is always in direct proportion to the activation energy at corresponding conversion rate points. FIG. 6D, FIG. 6E, and FIG. 6F show thermodynamic parameter values of the tea leaf sample Y2, FIG. 6G, FIG. 6H, and FIG. 6I show thermodynamic parameter values of the tea leaf sample Y3, and FIG. 6J, FIG. 6K, and FIG. 6L show thermodynamic parameter values of the tea leaf sample Y4. At the initial stages of the reactions of the tea leaf sample Y2, the tea leaf sample Y3, and the tea leaf sample Y4, αH is less than 0, indicating that the reaction is an exothermic reaction at this point, and the Rizhao green tea leaf sample Y1 has a sharp contrast with it.

The average values of ΔG of the Rizhao green tea leaf sample Y1 evaluated using the Kissinger, Ozawa, and Starink methods are 2058.33 KJ/mol, 2044.94 KJ/mol, and 2059.58 KJ/mol, respectively. It can be sure that the reaction in the whole pyrolysis process is non-spontaneous and needs extra energy input. The lower the value of ΔG, the less the energy provided by the reaction. As the temperature rises, the value of ΔG increases constantly. As can be seen from FIG. 6B, FIG. 6E, FIG. 6H, and FIG. 6K, the values of ΔG of the Rizhao green tea leaf sample Y1 at different a are all greater than those of the tea leaf sample Y3 and the tea leaf sample Y4. Within the interval 0.1<α<0.4, the value of ΔG of the Rizhao green tea leaf sample Y1 is greater than that of the tea leaf sample Y2, and within the interval 0.5<α<0.9, the value of ΔG of the Rizhao green tea leaf sample Y1 is less than that of the tea leaf sample Y2. This is consistent with the conclusion with respect to the pre-exponential factor.

The average values of ΔS of the Rizhao green tea leaf sample Y1 evaluated using the Kissinger, Ozawa, and Starink methods are 630.59 KJ/mol, 626.68 KJ/mol, and 630.96 KJ/mol, respectively. Greater ΔS indicates that the tea leaf particles has great randomness during pyrolysis. Apparently, in the whole pyrolysis process, ΔS is positive, and as the temperature rises, the value of ΔS increases constantly. Within the range 0.1<α<0.2, ΔS of the Rizhao green tea leaf sample Y1 exhibits a down trend, indicating that the disorder degree of the system within this interval decreases slightly. Within the range 0.2<α<0.9, ΔS exhibits an uptrend, indicating that the disorder degree of the system increases constantly with increasing temperature. This is consistent with the conclusion with respect to the kinetic parameter Ea value.

S7, an activation energy Ea prediction model for Rizhao green tea is established.

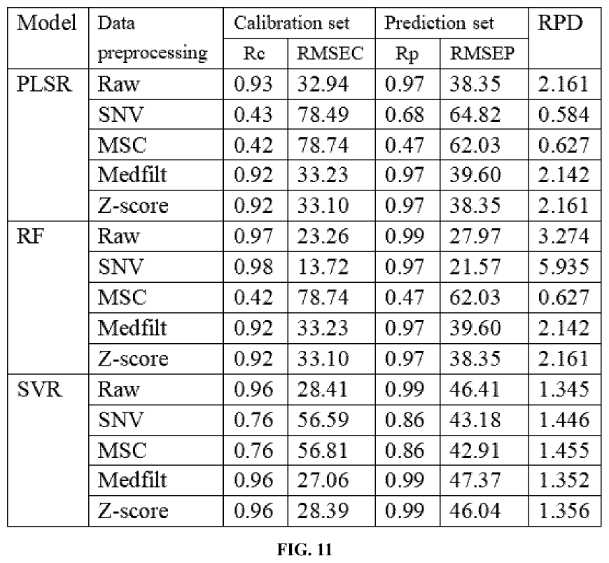

By the above steps of the present disclosure, an activation energy Ea value of the Rizhao green tea leaf sample Y1 at each conversion rate α is calculated, and linear partial least squares regression (PLSR) and nonlinear support vector regression (SVR) and random forest (RF) regression prediction models are established with three different physical parameters, namely different conversion rates α and different temperatures T and temperature rise rates β corresponding to the different conversion rates α, as inputs to the models. Before modeling, since the input three features are different physical quantities, they are unified to a same standard using four different preprocessing techniques so as to optimize prediction models. Different prediction models under different preprocessing algorithms established for the Ea value are as shown in FIG. 11. The RF model established based on SNV preprocessing has the best precision. A scatter diagram of a training set and a prediction set is plotted, as shown in FIG. 11. The value of Rc is 0.98, the value of Rp is 0.97, and the value of RPD is 5.935, indicating that the RF model established based on SNV preprocessing has strong prediction capability.

The present disclosure specifies the thermogravimetric curve spectrograms and the differential thermogravimetric curve spectrograms of the tea leaf samples of different producing areas, and results indicate that there are obvious differences in combustion characteristics in the combustion process between the Rizhao green tea leaf sample Y1 and the green teas of other producing areas. Compared with the other three tea leaf samples, the Rizhao green tea leaf sample Y1 has high initial temperature, slow decomposition, low cut-off temperature, high decomposition speed at the major thermal decomposition stage. The three-dimensional spectrograms of smokes generated by the tea leaf samples of different producing areas in the thermogravimetric process are specified, and results indicate that the Rizhao green tea leaf sample Y1 has obvious differences in the absorption peak and the absorption peak size of the three-dimensional spectrogram from other tea leaf samples, and has higher CO₂ intensity than other tea leaf samples in the range from 2229 cm⁻¹ to 2404 cm⁻¹. The generation of SO₂ is detected within the range of 1383 cm⁻¹, which is due to the decomposition of sulfur-containing organic and inorganic compounds in the samples. Others absorption peaks represent HCN (674 cm⁻¹), NO (1741 cm⁻¹), C═C (1440 cm⁻¹ to 1587 cm⁻¹), CO (2185 cm⁻¹), and HCl (2935 cm⁻¹), respectively, and these chemical substances are major gaseous products in the combustion process. The kinetic parameters and the thermodynamic parameters of the Rizhao green tea leaf sample Y1 and other tea leaf samples are specified. At all the stages of the pyrolytic reaction, the Rizhao green tea leaf sample Y1 has higher Ea than the tea leaf sample Y3 and the tea leaf sample Y4. Within the range 0.1<α<0.5, the Rizhao green tea leaf sample Y1 has higher Ea than the tea leaf sample Y2. Within the range 0.6<α<0.8, the Rizhao green tea leaf sample Y1 has lower Ea than the tea leaf sample Y2. At the later stage of the reaction, the Rizhao green tea leaf sample Y1 has higher Ea than the tea leaf sample Y2. This shows that the whole pyrolysis process exhibits an extremely complex reaction mechanism. A prediction model is established for the activation energy Ea of a Rizhao green tea leaf sample Y1, and an input to the prediction model is merely three characteristic parameters. The problem of model complexity is fundamentally solved. The established different regression models have extremely high prediction precision and can effectively predict the activation energy EA value of an unknown Rizhao green tea leaf sample and identify whether the green tea is the Rizhao green tea according to the value of the activation energy Ea.

What is claimed is:

1. A method for identifying a producing area of Rizhao green tea, comprising the following steps:
S1, collection and preprocessing of tea leaf samples:
collecting a Rizhao green tea leaf sample, a first tea leaf sample from a first province, a second tea leaf sample from a second province, and a third tea leaf sample from a third province, and preliminarily grinding the tea leaf samples, wherein the tea leaf samples are curled green teas;
S2, thermogravimetric analysis-Fourier transform infrared spectroscopy (TGA-FTIR) coupled testing:
performing the TGA-FTIR coupled testing using a thermal analyzer; before testing, levelling a balance of the thermal analyzer, weighing 10 mg±0.5 mg test sample in a weighing area of the balance, and then turning on the thermal analyzer for testing;
S3, infrared spectrogram analysis:
plotting three-dimensional spectrograms of smokes generated by the tea leaf samples in a pyrolysis process, and two-dimensional spectrograms at maximum decomposition rates in the pyrolysis process;
S4, thermogravimetric-differential thermogravimetric (TG-DTG) spectrogram analysis:
processing thermogravimetric data obtained from the TGA-FTIR coupled testing, importing temperatures and the thermogravimetric data in Origin software, performing a derivation on the thermogravimetric data to obtain differential thermogravimetric data, and plotting a TG-DTG curve spectrogram of the tea leaf samples with the temperatures as a horizontal axis and the thermogravimetric data and processed differential thermogravimetric data as a vertical axis;
S5, estimation of kinetic parameters of the Rizhao green tea:
estimating an activation energy using integration methods comprising Kissinger method, Ozawa method, and Starink method:
(1) the Kissinger method:
a calculation equation of the Kissinger method being as follows:
$$ln ( \frac{β}{{T}^{2}} ) = ln ( \frac{AR}{E} ) - \frac{E}{RT} \qquad ( 1 )$$
wherein β represents a temperature rise rate, in units of K/min; T represents a characteristic temperature, in units of K; E represents the activation energy, in units of KJ/mol; R represents a Boltzmann gas constant, with a value of 8.314, in units of J/(mol·K); A represents a pre-exponential factor; and at different temperature rise rates, E is determined from a linear curve slope of
$$ln ( \frac{β}{{T}^{2}} ) to - \frac{1}{T} ;$$
(2) the Ozawa method:
a calculation equation of the Ozawa method being as follows:
$$ln ( β ) = ln ( \frac{AEa}{R g ( α )} ) - 5.331 - 1.052 \frac{Ea}{RT} \qquad ( 2 )$$
wherein at different temperature rise rates, Ea, the activation energy, is determined by a linear curve slope of ln(β) to 1/T.
(3) the Starink method:
a calculation equation of the Starink method being as follows:
$$ln ( \frac{β}{{T}^{1.92}} ) = lg ( \frac{AR}{Rg ( α )} ) - 0.312 - 1.0008 \frac{Ea}{RT} \qquad ( 3 )$$
wherein the pre-exponential factor A is determined by the Kissinger method, and expressed as:
$$A = \frac{β Ea exp ( \frac{Ea}{RT} )}{{RT}^{2}} \qquad ( 4 )$$
estimating key thermodynamic parameters comprising enthalpy change ΔH, gibbs free energy change ΔG, and entropy change ΔS by the following equations:
$$Δ H = Ea - {RT}_{m} \qquad ( 5 )$$
$$Δ G = Ea + {RT}_{m} ln ( \frac{{k}_{b} {T}_{m}}{hA} ) \qquad ( 6 )$$
$$Δ S = ( Δ G - Δ H ) / {T}_{m} \qquad ( 7 )$$
wherein k_{b} represents a Boltzmann constant, a value of k_{b} is 1.38×10⁻²³ J/K; h represents a Planck constant, a value of h is 6.626×10⁻³⁴ J·s;
α represents a weight loss fraction or a thermal conversion rate, and a calculation equation of a is as follows:
$$α = ( {m}_{0} - m ) / ( {m}_{0} - {m}_{F} ) \qquad ( 8 )$$
wherein m₀ represents an initial mass of a sample, m represents a mass of a pyrolyzed sample at a given moment, and mf represents a final residue mass after thermal decomposition;
when the temperature rise rate β is constant,
$$β = \frac{d α}{dT} = \frac{d α}{dt} \frac{dt}{dT} \qquad ( 9 )$$
obtaining basic data of pyrolysis of the tea leaf samples at different thermal conversion rates α (a value range of α being 0.1 to 0.9, with an increment of 0.1), performing linear fitting by the Kissinger method, the Ozawa method, and the Starink method at different heating rates, respectively, and at each thermal conversion rate α, calculating corresponding activation energy and pre-exponential factor and plotting a point plot;
S6, calculation of pyrolysis parameters of the Rizhao green tea:
calculating the thermodynamic parameters enthalpy change ΔH, gibbs free energy change ΔG, and entropy change ΔS at each thermal conversion rate; and
S7, establishment of an activation energy Ea prediction model for the Rizhao green tea:
calculating an activation energy Ea value of the Rizhao green tea leaf sample at each thermal conversion rate α, establishing linear partial least squares regression and nonlinear support vector regression and random forest regression prediction models with physical parameters as inputs, and selecting an optimal Ea prediction model, wherein the physical parameters comprise different thermal conversion rates α, different characteristic temperatures T, and temperature rise rates β corresponding to the different thermal conversion rates α.

2. The method for identifying the producing area of the Rizhao green tea according to claim 1, wherein the step S1 comprises: sieving ground tea leaf samples through a 150-mesh screen, and selecting tea leaf particles below the 150-mesh screen as final test samples.

3. The method for identifying the producing area of the Rizhao green tea according to claim 2, wherein the step S2 comprises: before turning on experimental apparatuses, setting basic parameters of a thermogravimetry-Fourier transform infrared spectrometer, setting a test atmosphere to a high-purity nitrogen atmosphere, setting a flow velocity to 60 mL/min, setting a heating temperature range of the thermogravimetric analysis to be from room temperature to 800° C., and setting the temperature rise rate β to 10° C./min, 20° C./min, and 30° C./min at the thermogravimetric analysis.

4. The method for identifying the producing area of the Rizhao green tea according to claim 3, wherein the step S4 comprises: performing signal smoothing on the differential thermogravimetric data, selecting an adjacent average method, and setting a number of window points to 20, thereby obtaining the processed differential thermogravimetric data.

5. The method for identifying the producing area of the Rizhao green tea according to claim 4, wherein the pyrolysis process of the Rizhao green tea is mainly divided into three stages:
at a first stage, the temperature is within a range from room temperature to 192° C., and a weight of the Rizhao green tea decreases slowly with a mass loss of about 4%, which mainly results from loss of free moisture in a tea leaf caused by evaporation;
at a second stage, the weight loss of the tea leaf is maximum; an active pyrolysis zone is created; a temperature range of the second stage is from 192° C. to 501° C.; the mass loss of the Rizhao green tea is 55.4%; and in an interval, fragrant constituents in the tea leaf are decomposed and volatilized, and lignin, cellulose, and hemicellulose undergo violent pyrolytic reaction due to temperature rise, and are gradually carbonized, generating a large amount of gas; and
at a third stage, which is a carbonization stage within a range above 501° C., ash and charcoal are left as a residue is decomposed slowly at the third stage; inorganic ash is volatilized and decomposed; a pyrolysis of the lignin occurs within a wide temperature range of 192° C. to 800° C.; a DTG curve after 501° C. is results of lignin decomposition, and only slight mass loss occurs in a range of the DTG curve.

6. The method for identifying the producing area of the Rizhao green tea according to claim 5, wherein the step S7 comprises: before modeling, unifying, using four different preprocessing techniques, different thermal conversion rates α, characteristic temperatures T, and temperature rise rates β to a same standard so as to optimize the linear partial least squares regression and nonlinear support vector regression and random forest regression prediction models.

7. The method for identifying the producing area of the Rizhao green tea according to claim 6, wherein in the step S7, the four different preprocessing techniques are standard normal variate (SNV) preprocessing, multiplicative scatter correction (MSC), Medfilt, and normalization and standardization (Z-score).

8. The method for identifying the producing area of the Rizhao green tea according to claim 7, wherein a prediction model having maximum precision is selected according to a value of Rc, a value of Rp, and a value of RPD as the random forest regression prediction model established through the SNV preprocessing.
